(19) United States
(12) Patent Application Publication
(10) Pub. No.: US 2020/0276390 A1
(43) Pub. Date: Sep. 3, 2020

(54) E-CONNECTED AUTO-INJECTORS

(71) Applicant: MEDIMMUNE, LLC, GAITHERSBURG, MD (US)

(72) Inventors: MICHAEL C. SONG, GAITHERSBURG, MD (US); JANARDHANAN ANAND SUBRAMONY, GAITHERSBURG, MD (US)

(21) Appl. No.: 16/605,304

(22) PCT Filed: Apr. 19, 2018
(86) PCT No.: PCT/US2018/028292

§ 371 Date: Oct. 15, 2019

Related U.S. Application Data
(60) Provisional application No. 62/487,014, filed on Apr. 19, 2017.

(51) Int. Cl. A61M 5/20

(52) U.S. Cl. CPC A61M 5/2033; A61M 2205/3368; A61M 2205/3317; A61M 2005/2073; A61M 2205/8206; A61M 2205/3546; A61M 2205/3584; A61M 2205/50

(57) ABSTRACT

A device configured to delivery medication is disclosed. The device contains a plurality of sensors, including a magnetic proximity sensor and a temperature sensor. The proximity magnetic sensor can detect whether all the medication has been injected into a patient. The temperature sensor can ascertain whether the temperature of the medication has reached a predetermined or proper level for injection. The device also contains a locking device that can lock the device when the temperature of the medication is below this proper temperature and can automatically unlock the device when the temperature reaches or exceeds this proper temperature.

### FIELD OF THE INVENTION

The present invention relates to smart autoinjectors and/or autoinjectors that are connected to the Internet or to computing devices, such as smart phones, computer tablets, laptops and desktops, and that have an array of sensors to ascertain metrics for the autoinjectors in real-time and due to its connectivity these metrics can be read by the computing devices and/or transmitted via the Internet to be read by other computing devices.

### BACKGROUND OF THE INVENTION

Medical insurers are moving away from a unit priced payment model toward a more outcome based compensation model. Enabling medical device digital connectivity would provide valuable added information for patient, healthcare providers, doctors, pharmaceutical companies and payers.

An additional problem is that drug viscosity varies with temperature; the colder the temperature the higher viscosity the drug becomes. Cold drugs with higher viscosity may negatively impact successful drug delivery and potentially cause patient discomfort. For this reason, many instructions for drug delivery instruct the patient to let the drug warm to a proper or predetermined temperature, such as room temperature or body temperature, before injection to prevent cold injection.

There remains a need for a medicine delivery system that can monitor the injection of the medication and that can prevent the delivery system from activation when the temperature of the medication reaches the proper or predetermined temperature.

### SUMMARY OF THE INVENTION

One aspect of the present invention relates to capturing usage information obtained by magnetic proximity sensors integrated into the inventive autoinjector.

The present invention also captures and utilizes drug product temperature data to prevent device use, when the drug products have not reached proper temperature by locking the autoinjector, to prevent injection of below proper temperature medication into the patients. When the device and drug product reach desired injection temperature, the device would be automatically unlocked. The patients and/or healthcare provider may manually override this locking feature to inject the medication at any time.

While autoinjectors are used here as example to demonstrate the invention, the invention described in this document can be applied to any drug delivery devices with mechanical or electromechanical internal moving parts. The term “medication” used herein include, but is not limited to, medicines, vaccines, and any liquids that can be injected into human and animal patients.

The present invention relates to a device configured to delivery medication. The device contains a plurality of sensors, including a magnetic proximity sensor and a temperature sensor. The proximity magnetic sensor can detect whether all the medication has been injected into a patient. The temperature sensor can ascertain whether the temperature of the medication has reached a predetermined or proper level for injection. The device also contains a locking device that can lock the device when the temperature of the medication is below this proper temperature and can automatically unlock the device when the temperature reaches or exceeds this proper temperature.

### BRIEF DESCRIPTION OF THE DRAWINGS

In the accompanying drawings, which form a part of the specification and are to be read in conjunction therewith and in which like reference numerals are used to indicate like parts in the various views:

FIG. 1A is a cut-away exploded view of a conventional autoinjector; FIG. 1B is a partial cutaway view of FIG. 1A showing a prefilled syringe with a temperature sensor and the cut-away cover sleeve;

FIG. 2A is an exploded view showing the autoinjector and e-connected adaptor; FIG. 2B shows an assembled version of FIG. 2A;

FIG. 3A is a plan view of an autoinjector with embedded wires and other electrical connectors in an unconnected configuration; FIG. 3B shows the autoinjector of FIG. 3A in the connected configuration;

FIG. 4A shows internal components of the autoinjector including a magnetic sensor positioned on the firing pin, the firing spring and the cover lock; FIG. 4B is a perspective view of the firing pin;

FIGS. 5A-5B are cut away view of the autoinjector with a magnetic sensor showing the device in a pre-activation configuration and post-activation configuration, respectively;

FIG. 6A is a perspective view of the distal end of the lock sleeve; FIG. 6B plan views of the lock sleeve as it is assembled with interacting component shown with and without the firing spring present and shown with the magnetic sensor and/or temperature sensor possible placement location;

FIG. 7 is a plan view of the lock sleeve with at least one permanent magnet inserted therein;

FIG. 8 is a plan view of the body of the autoinjector with a Hall Effect type sensor adhered thereto;

FIG. 9 shows graphs of the magnetic signals from the sensors of FIGS. 7 and 8;

FIG. 10 shows graphs showing the magnetic signals from the sensors of FIGS. 7 and 8 with the permanent magnets in FIG. 7 assembled with opposite polarity;

FIG. 11A is a perspective view of the lock sleeve with a locking tab; FIG. 11B is an enlarged view of FIG. 11A; FIG. 11C is a partial, cross-sectional view of FIG. 11B showing the locking tab;

FIG. 12A is a perspective view of the cover sleeve with the locking tab; FIG. 12B is an enlarged, partial view of the locking tab of FIG. 12A; FIG. 12C is a cross-sectional view of the locking tab in FIGS. 12A-B with a portion of the body of the autoinjector and the syringe;

FIG. 13A an exploded view of the inventive adaptor showing the firing pin, the firing spring and cap; FIG. 13B is an enlarged view of the firing pin showing a locking slot and FIG. 13C is a further enlarged view of the locking slot; FIG. 13D is an exploded view of the adaptor's cap with the rotatable turn lock and a top view of the cap; and FIG. 13E is a perspective view of the turn lock; FIG. 13F is a side view of a rotatable fork to rotate the turn lock;

FIG. 14A is a side cut-away view of the autoinjector with another embodiment of the locking mechanism; FIG. 14B is an end view showing the firing pin latch and the firing pin latch channel;

FIG. 15A is a side, cut-away view of another embodiment of the locking mechanism incorporating a solenoid-type actuator; FIG. 15B is an enlarged view of the firing mechanism latch and lock; and FIG. 15C is a side view of FIG. 15B;

FIG. 16 is an exploded view of a conventional autoinjector.

### DETAILED DESCRIPTION OF THE PREFERRED EMBODIMENTS

An autoinjector, as illustrated in FIG. 16, is a drug delivery device with a stored power for injection type delivery of drugs stored within prefilled syringe 94. The autoinjector contain a cap 96 that enables the end user to remove the enclosed prefilled syringe's rigid needle shield 97. Once cap 96 is removed, the autoinjector can be fired whereby the stored energy within the power unit 95 is released. When the autoinjector is activated by depressing the Cover Sleeve 34, the Cover Sleep pushes up the Lock Sleeve 36 which allow the power unit to release the firing pin lock 99 holding the firing pin in place. The release of the firing pin lock 99 allows the firing pin spring 100 to push the firing pin forward against the syringe plunger. (See FIG. 17) The stored energy is directed against the plunger rod/firing pin 98, which pushes against the prefilled syringe's plunger 99 expelling the drug content. FIG. 16 provides a pictorial representation of the autoinjector and prefilled syringe 94 prior to assembly. The autoinjector generally comprises power unit 95 and front shield 93 with cap 96 attached.

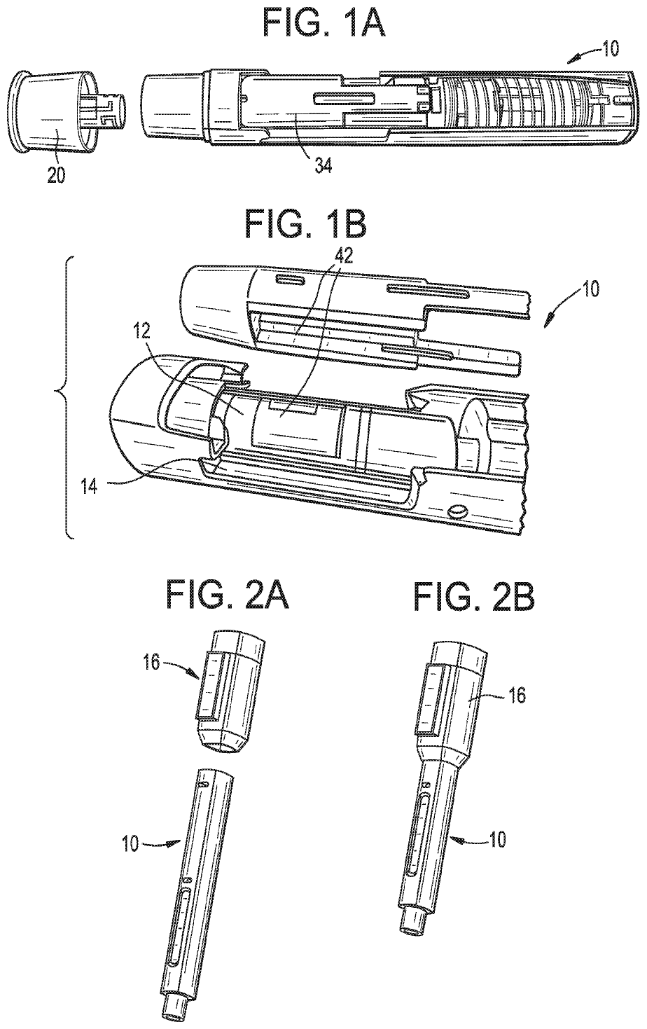

In one embodiment, the inventive autoinjector is sensor rich, and is connected to computing devices, such as smart phones, computing tablets, laptop and desktop computers, as well as mainframe computers or servers and storage clouds through direct connection or through connection to the Internet. In one embodiment, as shown in FIGS. 1A and 1B, certain autoinjectors 10, such as a 2.25 ml autoinjector, do not have sufficient internal space to accommodate the sensors. As shown, in the enlarged view of FIG. 1B, a prefilled syringe 12 is typically inserted into autoinjector 10 and remaining internal space 14 is used as a window viewing area. Preferably, the sensors are located in an adaptor 16 or are connected to adaptor 16, as best shown in FIGS. 2A and 2B, and is sized and dimensioned to be removably attached to autoinjector 10.

Adaptor 16 may include a number of electrical and display components, such as but not limited to a digital display, sensors, such as position, acoustic, and vibration sensors, a microprocessor, a storage memory such as flash memory, NFC detector, contact switches and Bluetooth transmission system. The sensors would detect device usage information, such as internal component movement (e.g., via magnetic position sensors), device firing sound and vibration (e.g., acoustic and vibration sensors). The NFC detector can be utilized to detect device unique information that can be embedded within the device label via an NFC chip. The microprocessor will process the gathered sensor and other input data (e.g., data from NFC embedded label) and store said information onto the storage memory until ready for transmission via the Bluetooth or other transmission system.

Adaptor 16 may also have a power source, such as a battery or solar panel, and a DC motor or solenoid valve, labeled as element 100 hereinafter, to provide rotational or translational movements within adaptor 16 and/or autoinjector 10 to move the device between an interfering configuration, in which the operation of autoinjector 10 is blocked or restricted, and a non-interfering configuration, in which the operation of autoinjector 10 is operable.

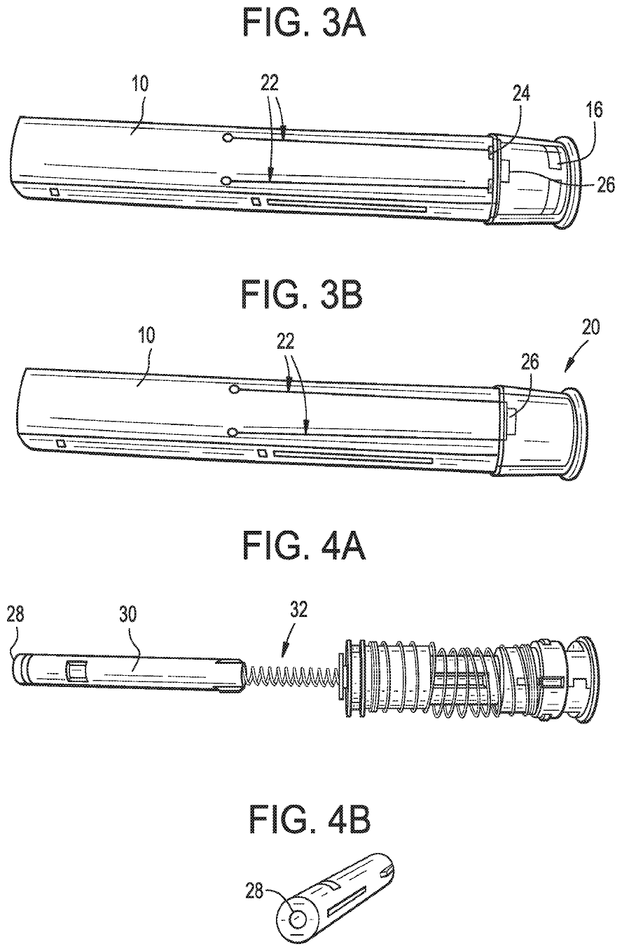

In one embodiment, the present invention is capable of determining when adaptor 16 is attached and then removed from autoinjector 10. As best shown in FIGS. 3A and 3B, autoinjector 10 in this embodiment has two electrical wires 22 that are embedded in the wall of the housing of the autoinjector. The proximal ends of these wires are spaced apart to form an open gap 24, as best shown in FIG. 3A. Adaptor 16 comprises a conductive strip or bridge 26 that is sized and dimensioned to bridge the proximal ends of wires 22, so that when the adaptor is fully inserted onto autoinjector 10, conductive strip 26 connects the proximal ends of wires 22 to close the circuit formed by the circuitry in adaptor 16, wires 22 and bridge 28.

The removal of adaptor 16 would open this circuit, which is readable by adaptor 16, and the insertion of adaptor would close this circuit, which is also readable by adaptor 16 and indicates that adaptor 16 is operational. The microprocessor in adaptor 16 can transmit this information to connected computing devices and/or stores this information locally in a memory inside adaptor 16.

Adaptor 16 of the present invention may also detect whether a medication delivery was completed and whether a delivery error had occurred. Heretofore, patient vigilance by viewing the viewing window after delivery is relied on to confirm whether the delivery was completed or successful. To relieve the patient or health care provider from this task, the present inventors are adapting a magnetic proximity sensor to adaptor 16 and autoinjector 10. Magnetic proximity sensors generally comprise two magnetic components. When these components are located proximate to each other, their magnetic fields affect each other and the effects can be measured and the distance between the two components can be derived. Magnetic proximity sensors would detect a strong signal when their two components are near each other, and a weak signal or no signal when the two components are distant from each other. An example of magnetic proximity sensors includes, but is not limited to, a Hall Effect sensor disclosed in www.electronics-tutorials.ws/electromagnetism/hall-effect.html, and in U.S. Pat. No. 7,698,936, which are incorporated herein by reference in its entirety.

Referring to FIGS. 4A-B, one magnetic proximity sensor 28 is attached to or adhered to the distal end of firing pin 30, which is biased by firing spring 32. The other proximity sensor (not shown) is housed within adaptor 16 and connected to its circuitry and microprocessor. Due to the space constraint, namely space 14 within autoinjector 10 taken by firing pin 30 and firing spring 32, magnetic sensor 28 is located at the distal end of firing pin 30. Prior to the deployment of firing pin 30 with firing spring 32 in a fully compressed state, magnetic sensor 28 is located proximate to the other proximity sensor within adaptor 16. In this configuration, the circuitry within adaptor 16 can detect magnetic sensor 28. When firing pin 30 is deployed successfully, magnetic sensor 28 should be located far from adaptor 16, such that the circuitry and the other half of the proximity sensor can no longer detect magnetic sensor 28 or detects only a weak signal. This would indicate a successful injection of the medication.

On the other hand, if the injection is incomplete and some of the medication remains in the syringe, then magnetic sensor 28 would remain close to adaptor 16 and the other magnetic sensor, and the circuitry would still detect magnetic sensor 28. The circuitry within adaptor 16 preferably would communicate a warning to the patient or healthcare provider, such as an audible alarm or visual signal, e.g., LED light.

To minimize potential damage to magnetic sensor 28, preferably it is at least partially embedded within the material of firing pin 30.

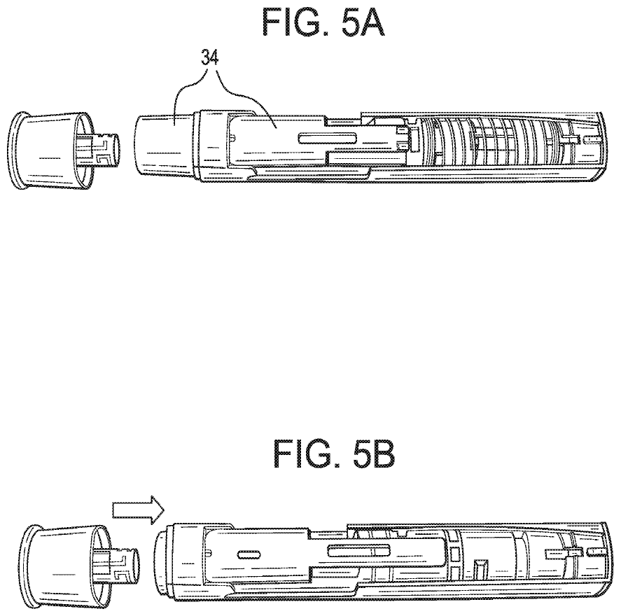

In another embodiment, magnetic sensor 28 can be placed on cover sleeve 34, as shown in FIG. 1. As best shown in FIGS. 5A and 5B, magnetic sensor 28 is positioned on the distal end of cover sleeve 34. FIG. 5A illustrates autoinjector 10 before activation or deployment and FIG. 5B illustrates autoinjector 10 after activation showing proximity magnetic sensor 28 to have moved distally along with cover sleeve 34. The embodiment of FIGS. 5A and 5B, otherwise, functions similarly to the embodiment of FIGS. 4A and 4B.

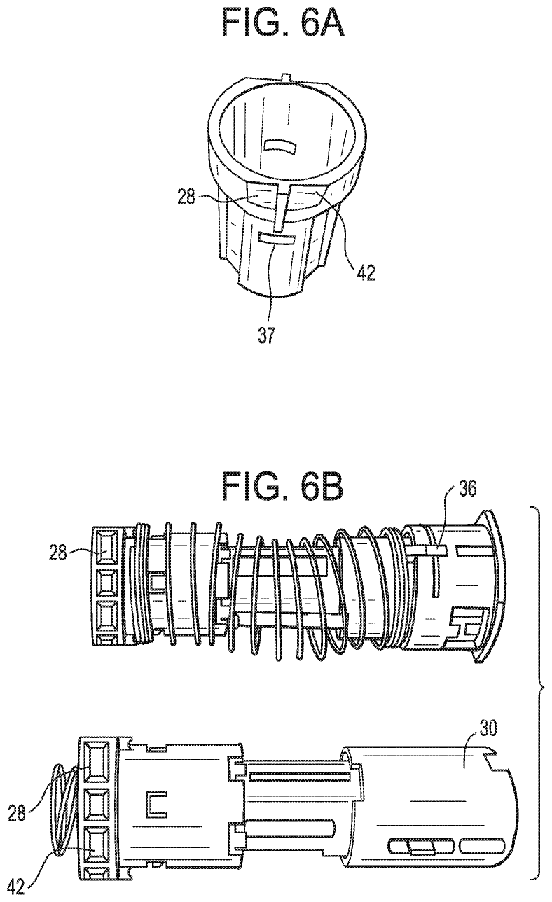

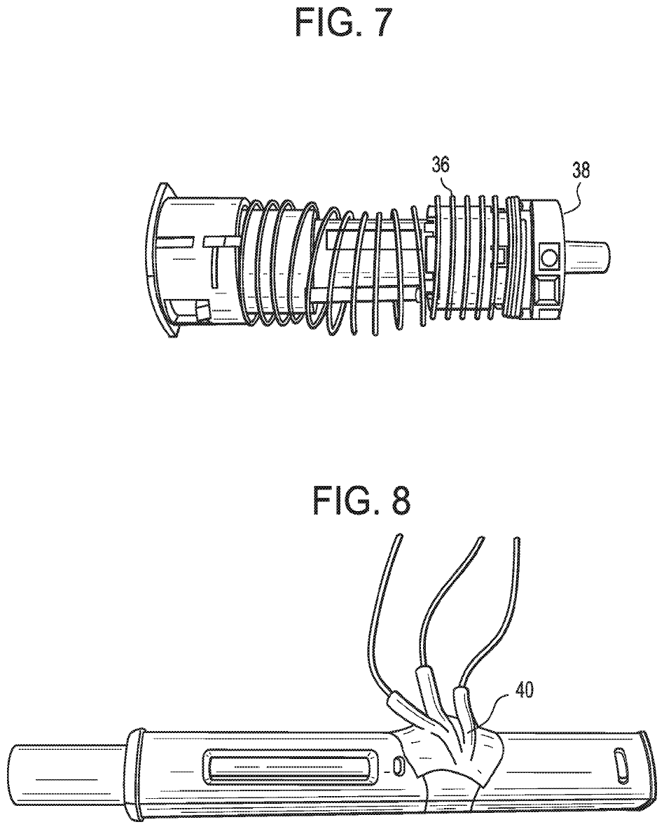

A variation of the embodiment of FIGS. 5A and 5B is shown in FIGS. 6A-6B, where magnetic sensor 28 is placed on lock sleeve 36. FIG. 6A shows end cap 35 of lock sleeve 36 with magnetic sensor 28 and/or temperature sensor 42 attached thereon. Since lock sleeve 36 moves when cover sleeve 34 moves, as shown in FIGS. 5A and 5B, locating magnetic sensor 28 on lock sleeve 36 functions in a similar fashion.

In accordance with another aspect of the present invention, the timing and the time duration of the injection of the medication can be measured with the magnetic proximity sensors or Hall Effect sensors described above. In this embodiment, both components of the magnetic proximity sensors are located on or within the body of autoinjector 10, as best shown in FIGS. 7 and 8. In this embodiment, at least one permanent magnet 38 in inserted or otherwise attached to the moving end of lock sleeve 36 of autoinjector 10. Preferably, Neodynium permanent magnet is used. This magnet is generally made from an alloy of Neodynium, iron and boron (NdFeB), and is available in sizes, e.g., 1/16 or 1/32 inch in thickness, that can fit into lock sleeve 36, as shown in FIG. 7. A single magnet 38 can be inserted or two magnets 38 with opposite poles oriented to each other can be used. As shown in FIG. 8, a single component 40 of a Hall Effect sensor is attached, e.g., taped or epoxied, to the body of autoinjector 10. FIG. 8 shows a prototype of this embodiment; a production version would have the component 40 either embedded to the body or permanently affixed thereto. The wires would also be embedded or permanently affixed to the autoinjector's body, and be connected to the circuitry and microprocessor in adaptor 16. Two Hall Effect sensors 40 can provide redundancy and accuracy.

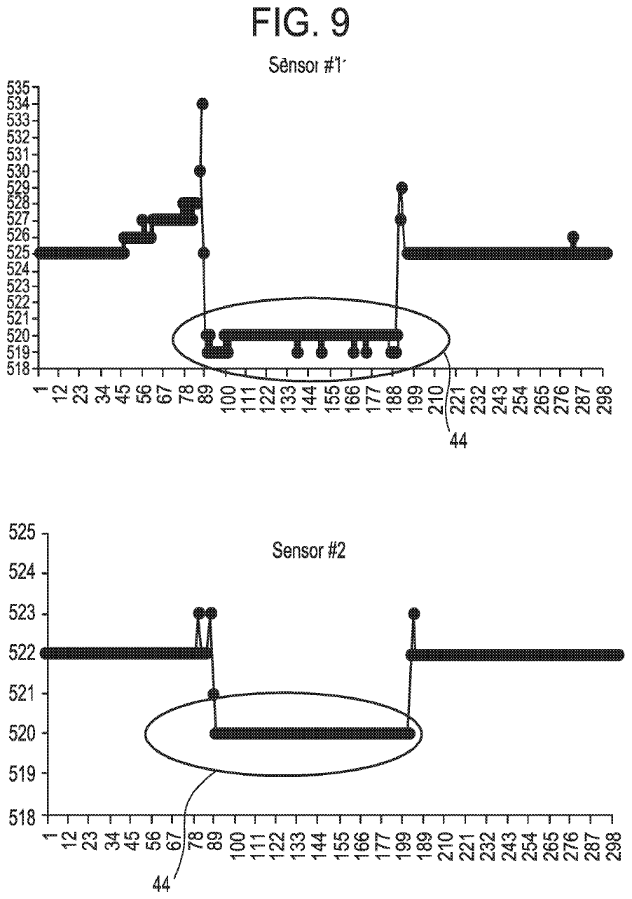

FIG. 9 shows the measured magnetic fields when a single permanent magnet 38 passes by two Hall Effect sensors 40 during activation. The horizontal axis represents a time axis and may commence recording when the patient/user activates the autoinjector. The detected magnetic fields by the Hall Effect sensors show an abrupt change when permanent magnet 38 passes by Hall effect sensor 40, as the medication is ejected. Both the timing and the time duration 44 of the injection can readily be extracted from the graphs in FIG. 9.

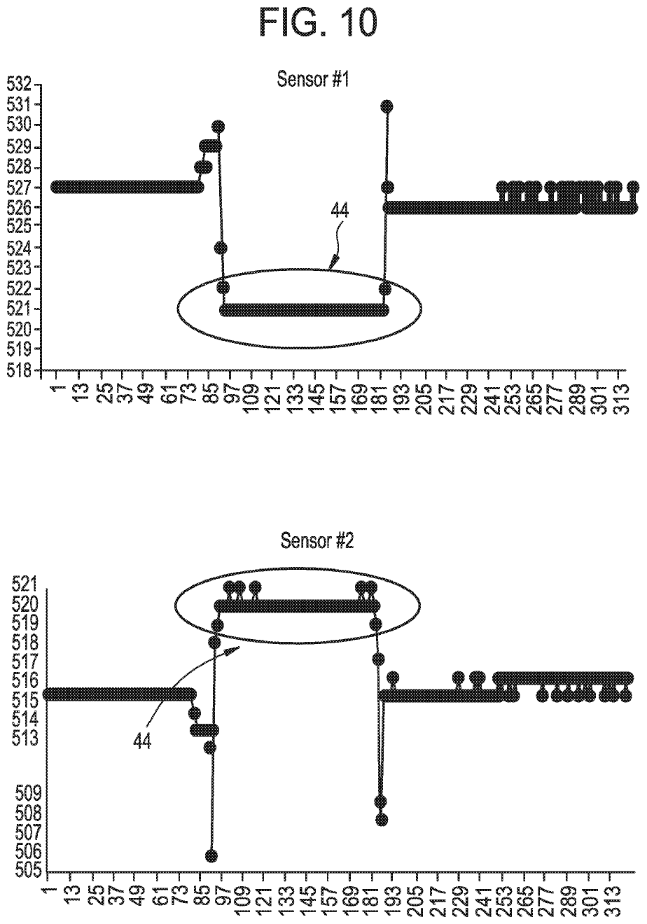

When two permanent magnets 38 with opposite polarity, as discussed above, are used and the two Hall Effect magnets 40 are placed on either side of the two permanent magnets 38, the detected magnetic fields are illustrated in FIG. 10. The opposite polarities produce two signals that are also opposite from each other and the user can ascertain the signal that corresponds with a particular permanent magnet.

When the patient or health care provider removes autoinjector 10 from the injection site, lock sleeve 36 would return passed its original position and be locked into place. This return motion would also be captured by the magnetic proximity sensor.

The embodiments shown in FIGS. 7-10 may also determine whether the medication was completely ejected from autoinjector 10 or from syringe 12 by evaluating the length of time duration 44 illustrated in FIGS. 9 and 10. If the graphs stop within the expected time duration segment 44, then the graphs indicate that firing pin 30 did not reach its expected destination. Additionally, if time duration segment 44 on the graphs in FIGS. 9 and 10 is shorter or longer than the expected duration, this may also indicate anomalies that are detectable by the circuitry and microprocessor in adaptor 16.

The embodiments shown in FIGS. 7-10 can also detect the movement of autoinjector 10, because this movement may transfer a slight motion through cover sleeve 34 to lock sleeve 36, and this motion can be picked up by the magnetic proximity sensor. Locking latches, discussed below, when released can also be detected by the magnetic proximity sensor.

In accordance to another aspect of the present invention, the present invention also includes a temperature sensing capability to measure the temperature of the medication to ensure that the medication reaches a predetermined or proper temperature, such as room temperature, body temperature, or another comfortable temperature, prior to being injected into the patient. The thermal sensor 42 can directly measure the temperature of prefilled syringe 12, which is typically refrigerated before use, by being attached to syringe 12 or to the inside of cover sleeve 34 which encloses syringe 12, as shown in FIG. 1B. Thermal sensor 42 can also indirectly measure this temperature by being attached to another component in autoinjector 10, such as lock sleeve 36 as shown in FIG. 6. In situation where thermal sensor 42 is not in direct contact with syringe 12, the circuitry and microprocessor in adaptor 16 can implement a time delay from when thermal sensor 12 reaches the target injection temperature and when injector 10 is unlocked to inject to take into account the differences in heat transfer and heat capacitance properties of the different materials inside autoinjector 10. Suitable temperature sensors include but are not limited to thermistors and thermocouples, such as those discussed in U.S. Pat. No. 7,698,936, and strain gages, etc.

In accordance with another aspect of the present invention, the temperature readings from thermal sensor 42 can be employed to lock autoinjector 10 to prevent activation before the pre-filled syringe reaches the proper temperature. The autoinjector 10 in one embodiment is automatically unlocked when the syringe temperature reaches the proper temperature. A user can also manually unlock autoinjector 10 if the event that an injection is necessary before the syringe reaches the proper temperature.

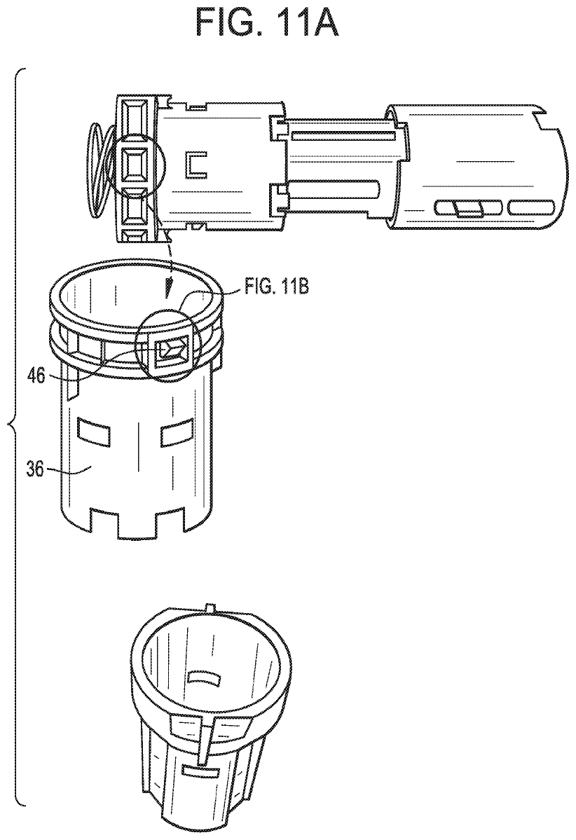

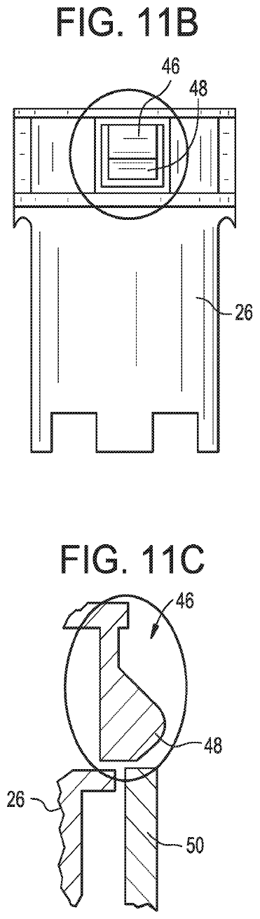

Referring to FIGS. 11A-C, lock sleeve 36 is provided with one or more locking tabs 46 connected at its proximal end to a lid of lock sleeve 36 in a cantilever manner, and has a free protruding end 48, which is sized and dimensioned to interfere with a wall 50 of the housing of autoinjector 10. Locking tab 46 acts like a live-hinge at its connection to the lid of lock sleeve 36 and protruding end 48 can be moved inward automatically, or pushed inward manually by a user to a non-interfering position with wall 50 to allow lock sleeve 36 to be actuated to eject the medication from syringe 12.

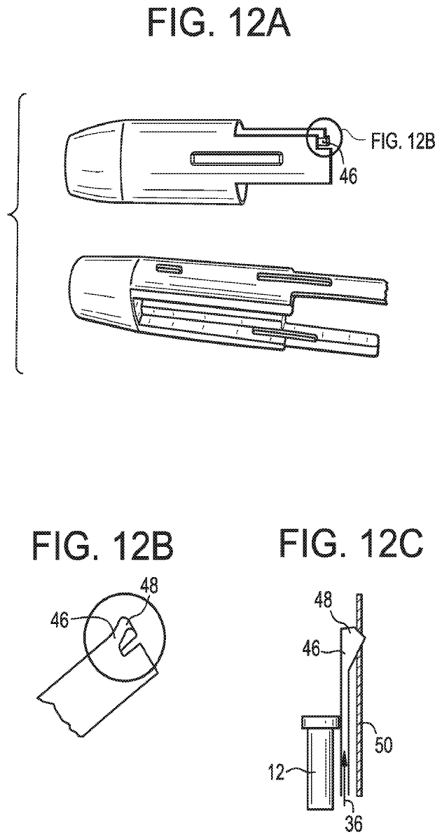

Alternatively, the one or more locking tab 46 can be positioned on cover sleeve 34, as best illustrated in FIG. 12. Locking tab 46 operates in the same or similar fashion when locating on cover sleeve 34 or lock sleeve 36.

In another embodiment, a weak, breakable string made from a shape memory alloy (SMA) connects protruding end 48 of locking tab 46 to a rigid, immovable portion of autoinjector 10 or adaptor 16. The SMA string has one shape, e.g., longer length at a certain lower temperature, e.g., temperature that the medications are refrigerated, and another shape, e.g., shorter length at a certain higher temperature, e.g., the proper, predetermined temperature for injection. In this embodiment, when the syringe's temperature rises to the proper temperature, the SMA string automatically lengthens to push protruding end(s) 48 inward allowing it to overcome wall 50. When a cooled or refrigerated syringe 12 is inserted into autoinjector 10, the SMA string automatically shortens allowing locking tab(s) 46 to flex to the interfering position. Suitable SMA materials include, but are not limited to, nickel-titanium or nitinol, which is commercially available as Flexinol™. Other suitable SMA materials include the alloys of Ag—Cd, Au—Cd, Cu—Al—Ni, Cu—Sn, Cu—Zn, Cu—Zn—X (X═Si, Al, Sn), Fe—Pt, Mn—Cu, Fe—Mn—Si, Pt alloys, Co—Ni—Al, Co—Ni—Ga, Ni—Fe—Ga, Ti—Pd in various concentrations, Ni—Ti—Nb and Ni—Mn—Ga.

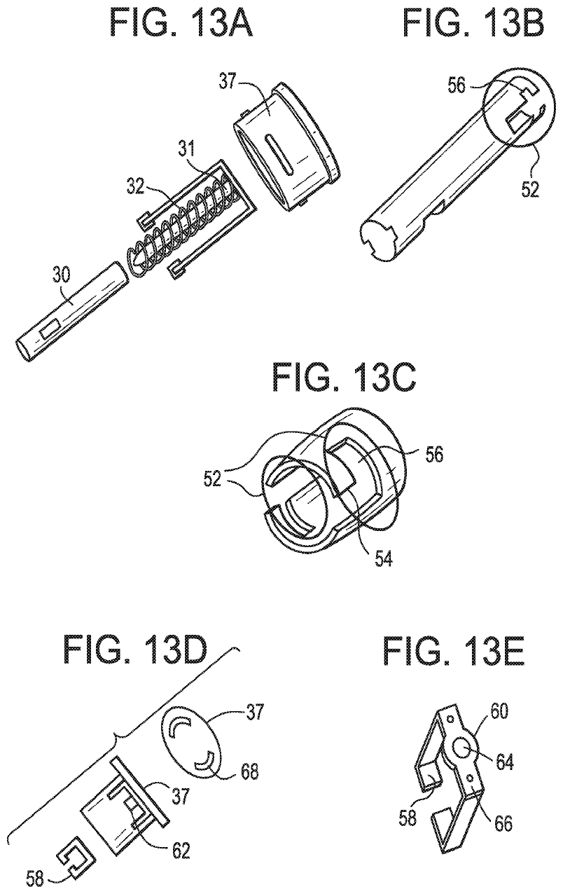

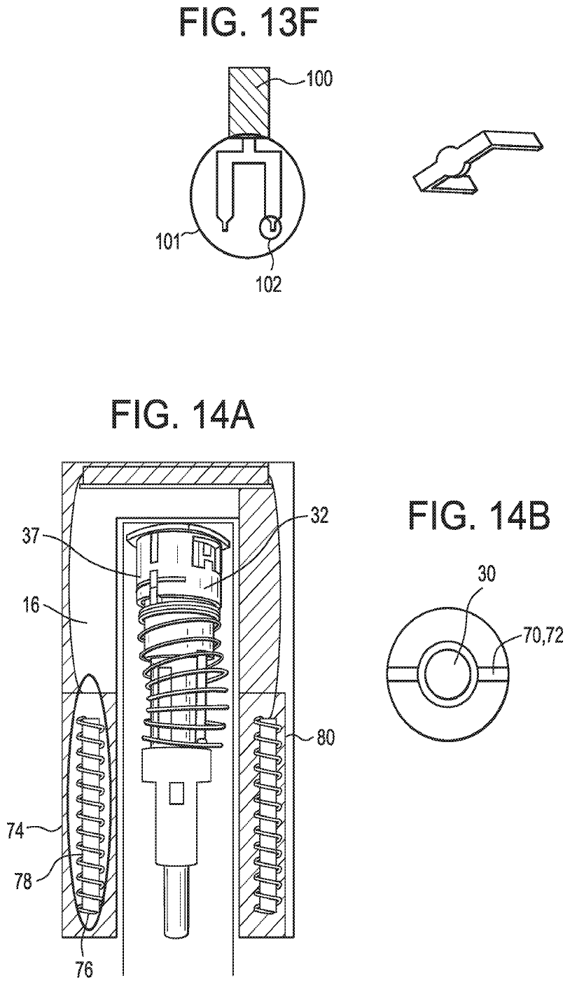

Another embodiment of the locking mechanism to be applied to firing pin 30 is shown in FIGS. 13A-E. FIG. 13A shows firing pin 30, firing spring 32, spring support 31 and end cap 37. FIGS. 13B-13C show at least one locking slot 52, which comprises a longitudinal slot 54 and at least one side slot 56, on the body of firing pin 30. Locking slot 52 may be a bayonet-type slot, and is sized and dimensioned to receive bent arms 58 of rotatable turn lock 60. As best shown in FIG. 13D, turn lock 60 resides within end cap 37 and is rotatably supported on pin 62, which is received by aperture 64 on top of turn lock 60. The top of turn lock 60 also have divots 66, which are accessible through curved openings 68 on top of end cap 37.

In a non-interfering configuration, i.e., firing pin 30 is free to activate and discharge medication from syringe 12, bent arms 58 of turn lock 60 are located in longitudinal slot 54. Turn lock 60 is rotatable, so that bent arms 58 are rotated into side slot 56 to place bent arms 58 in an interfering configuration by not allowing firing pin 30 to activate. A rotatable fork 101, as best shown in FIG. 13F, within adaptor 16, which is preferably attached to the DC motor or solenoid valve 100 discussed above, may be inserted through curved openings 68 and engage divots 66 via two pegs 102 on rotatable fork 101 to rotate turn lock 60 from the non-interfering configuration to the interfering configuration, depending for example on the readings of temperature sensor 42, as discussed above. In other words, when the temperature is at the proper injection temperature, bent arms 58 are positioned within longitudinal slot 54, and when the temperature is below the proper temperature, bent arms 58 are positioned within side slot 56.

Yet another embodiment of the locking mechanism is shown in FIGS. 14A-14B. As best shown in FIG. 14B, firing pin 30 is blocked from activation by at least one ferrous firing pin latch 70 restrained within latch channel 72. In an interfering configuration, two firing pin latches 70 pinch firing pin firing 30 thereby preventing it from deploying. The two firing pin latches 70 are sized and dimensioned to be partially inserted within two corresponding slots oriented about 180° from each other in the interfering configuration. Alternatively, firing pin latch 70 is positioned across firing pin 30 thereby preventing it from deploying. Due to its ferrous property, firing pin latch(es) 70 can be moved by an electromagnetic force, within latch channel 72. This magnetic force can be provided, as best shown in FIG. 14A, by one or more electromagnets 74. Electromagnet 74 may comprise a metallic, preferably ferrous, rod 76, wrapped by conductive coil 78. A magnetic force is generated when an electrical current flows through coil 78 to move firing pin latch 70. As shown, two electromagnets 74 are deployed to move two firing pin latches 70 pinching firing pin 30. The circuitry and microprocessor in adaptor 16 can selectively connect the adaptor's battery to conductive coil 78 to move firing pin latch 70 when the temperature of syringe 12 reaches the proper temperature, as discussed above.

Optionally, an electromagnetic shield 80, such as a Faraday cage, is deployed either to contain the electromagnetic field generated by electromagnet 74 or to isolate the circuitry and microprocessor in adaptor 16 from the electromagnetic field.

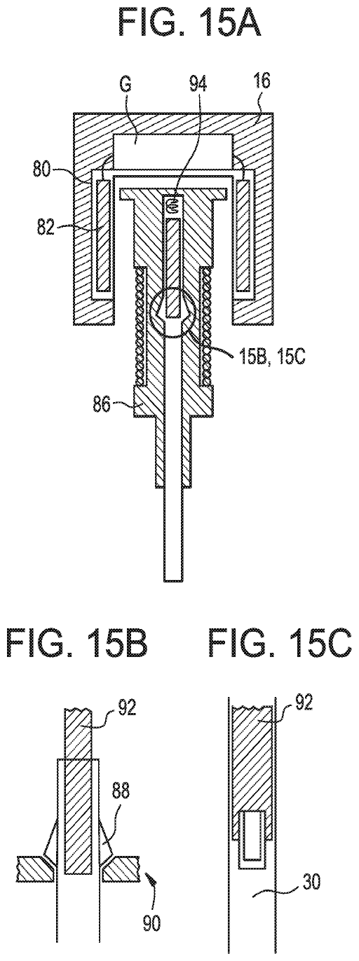

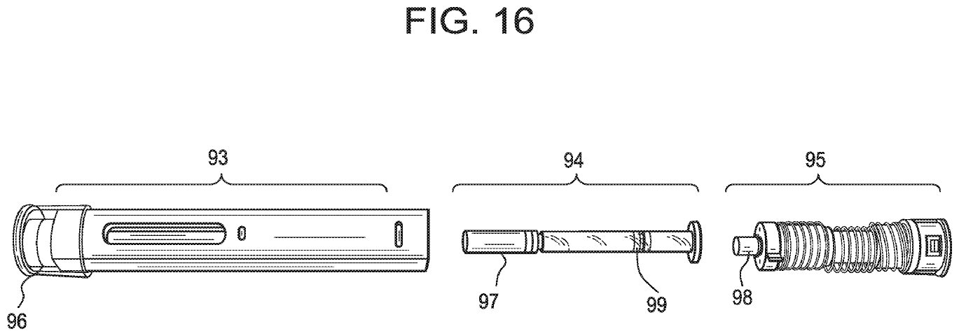

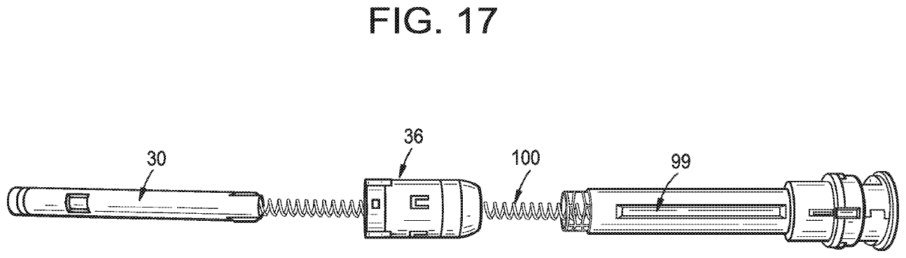

Another locking mechanism is shown in FIGS. 15A-C. In this embodiment, an electromagnetic coil 82 is placed within adaptor 16, as shown in FIG. 15A, which is electrically connected to the circuitry/microprocessor 84 in adaptor 16. Positioned within adaptor 16 is trigger mechanism 86, which moves upward to trigger firing pin 30 to move downward to activate autoinjector 10. This locking mechanism prevents trigger mechanism 86 from moving upward due to the interference between firing mechanism latch(es) 88 and firing mechanism lock(s) 90. As best shown in FIGS. 15B-15C, firing mechanism latch 88 are hinged tabs, similar to tabs 46 shown in FIGS. 11A-11C. The free end of firing mechanism latch 88 protrudes from firing pin 30 in a cantilevered fashion to create a live hinge connection, and interferes with firing mechanism lock 90, which is preferably connected to trigger mechanism 86. In a relaxed state, firing mechanism latch 88 would tuck within firing pin 30, and this embodiment would be in a non-interfering configuration, i.e., trigger mechanism 86 is free to move upward. A metal rod 92 is inserted within firing pin 30, as shown, and pushes firing mechanism latch 88 outward to interfere with firing mechanism lock 90. This metal, preferably ferrous, rod is maintained in this interfering configuration by a relatively weak spring 94.

When the temperature of syringe 12 reaches the proper injection temperature, the circuitry and microprocessor 84 would sense this temperature from thermal sensor 42 and would send a current from the battery within adaptor 16 to electromagnetic coil 82 in a direction that produces a magnetic field/force in the upward direction. Spring 94 is sized and dimensioned not to resist this magnetic force and metal rod 92 is pushed upward above firing mechanism latch 88. Latch 88 would revert to its relaxed state and move to the non-interfering configuration. Firing mechanism lock 90 can move upward passed firing mechanism latch 88 and trigger mechanism 86 can move upward to activate autoinjector 10.

Alternatively, adaptor 16 can send the current continuously through electromagnetic coil 82 to keep autoinjector 10 in the non-interfering configuration continuously and an electromagnetic shield 80 is deploy to contain the electromagnetic field, or adaptor 16 can send the current at the end of a predetermined time delay period, e.g., a few seconds, after trigger mechanism is activated.

All the embodiments described herein can be used in any drug delivery apparatus and the present invention is not limited to those described and/or illustrated herein.

While it is apparent that the illustrative embodiments of the invention disclosed herein fulfill the objectives stated above, it is appreciated that numerous modifications and other embodiments may be devised by those skilled in the art. Therefore, it will be understood that the appended claims are intended to cover all such modifications and embodiments, which would come within the spirit and scope of the present invention.

We claim:

1. A delivery device adapted to receive a pre-filled syringe containing a medication, wherein the delivery device comprises
an ejector that pushes the medication out of the pre-filled syringe when activated, and at least one magnetic proximity sensor that moves with the ejector, and
an adaptor configured to be removably attached to the delivery device adapted to read information obtained by the at least one magnetic sensor.

2. The delivery device of claim 1, wherein the adaptor comprises a circuitry and a microprocessor.

3. The delivery device of claim 2, wherein the adaptor further comprises a battery.

4. The delivery device of claim 1, 2 or 3, wherein the adaptor is connected to a computing device.

5. The delivery device of claim 4, wherein the adaptor is connected to the computing device through WiFi or Bluetooth.

6. The delivery device of claim 1 further comprising another magnetic sensor fixedly attached to a body of the delivery device, wherein said another magnetic sensor reads the at least one magnetic proximity sensor as the at least one magnetic proximity sensor moves relative to the another magnetic sensor.

7. The delivery device of claim 1 further comprising another magnetic sensor fixedly attached to the adaptor, wherein said another magnetic sensor reads the at least one magnetic proximity sensor as the at least one magnetic proximity sensor moves relative to the another magnetic sensor.

8. The delivery device of claim 6 or 7, wherein the at least one magnetic proximity sensor comprises at least one permanent magnet and wherein the another magnetic sensor comprises a Hall Effect sensor.

9. The delivery device of claim 1, wherein the at least one magnetic sensor is located on a firing pin, a cover sleeve or a lock sleeve of the delivery device.

10. A delivery device adapted to receive a pre-filled syringe containing a medication, wherein the delivery device comprises
an ejector that pushes the medication out of the pre-filled syringe when activated, and at least one temperature sensor that ascertains a temperature of the medication, and
an adaptor configured to be removably attached to the delivery device adapted to read the at least one temperature sensor, and
a locking device that is movable from an interfering configuration, where the ejector is prevented from activation, to a non-interfering configuration, where the ejector can activate,
wherein when the temperature of the medication is below a predetermined temperature the locking device is in the interfering configuration, and wherein when the temperature of the medication reaches or exceeds the predetermined temperature the locking device automatically moves to the non-interfering configuration.

11. The delivery device of claim 10, wherein the locking device comprises a hinged tab that interferes with a body of the delivery device in the interfering configuration.

12. The delivery device of claim 10, wherein the adaptor comprises a circuitry configured to move the locking device between the interfering configuration and the non-interfering configuration.

13. The delivery device of claim 12, wherein the circuitry comprises a DC motor or a solenoid valve, and a battery.

14. The delivery device of claim 12 or 13, wherein the circuitry further comprises a microprocessor.

15. The delivery device of claim 12, wherein the adaptor is connected to a computing device via WiFi or Bluetooth.

16. The delivery device of claim 12, wherein the adaptor further comprises at least one electromagnetic device, wherein when activated the electromagnetic device produces a magnetic field to move the locking device between the interfering configuration and the non-interfering configuration.

17. The delivery device of claim 10, wherein the locking device comprises a shape memory alloy element that moves the locking device between the interfering configuration and the non-interfering configuration.
